(19) United States
(12) Patent Application Publication
(10) Pub. No.: US 2019/0303648 A1
(43) Pub. Date: Oct. 3, 2019

(54) SMART SURVEILLANCE AND DIAGNOSTIC SYSTEM FOR OIL AND GAS FIELD SURFACE ENVIRONMENT VIA UNMANNED AERIAL VEHICLE AND CLOUD COMPUTATION

(71) Applicant: QRI GROUP, LLC, Houston, TX (US)

(72) Inventors: Xiang Zhai, Sugar Land, CA (US); Kui Liu, Houston, TX (US); William J. Nash, Houston, TX (US); David Castineira, Cambridge, MA (US)

(21) Appl. No.: 16/373,053

(22) Filed: Apr. 2, 2019

Related U.S. Application Data
(60) Provisional application No. 62/651,404, filed on Apr. 2, 2018.

(51) Int. Cl. G06K 9/00; G06K 9/62; G10L 25/51; G01N 33/00

(52) U.S. Cl. CPC G06K 9/0063; G06K 9/00664; G06K 9/6256; H04N 5/247; G10L 25/51; G01N 33/0062; G06K 9/6262

(57) ABSTRACT

In accordance with various embodiments of the disclosed subject matter, a smart surveillance and diagnostics system for oil and gas field surface environment via unmanned aerial vehicle (UAV) and cloud computing are provided. Methods and systems provide various functionality including performing, by multiple GPUs or HPCs, a fast pair-wise registration process, a mask setting process, a background generation process, a foreground generation process using a parallel computation infrastructure, a deep learning classification process, and performing anomalism detection (including vision, acoustic and gas concentration) and 3D augmented reality and 2D Panorama view reconstruction under a variety of conditions.

### CROSS-REFERENCE TO RELATED APPLICATION

This application claims the benefit of U.S. Provisional Application No. 62/651,404, filed Apr. 2, 2018, which is incorporated by reference herein in its entirety.

### BACKGROUND

Currently, oil and gas field surveillance is largely performed by human operators. The human operators have to be on-site checking the surface facilities such as pipelines and pumps. Sometimes the terrain accessibility can be very challenging, since many oil and gas production sites are located in mountainous areas and other harsh environments caused by extreme hot (e.g. Western Texas, Middle East) or cold (e.g. Alaska, The North Sea) temperatures. Modern satellite imagery (such as Synthetic Aperture Radar) provides high-resolution and wide coverage images of the oil and gas field. However, the cost of purchasing commercial satellite images is expensive. Surveillance camera systems may be used to view an area, but the field coverage range is small. All of these situations call for an automated, economic and wide-coverage surveillance system.

Traditional UAV-based implementations used in the oil and gas industry mainly focus on geophysical survey, animal detection and pipeline tracing. As such, this type of UAV implementation cannot be directly generalized to mass surveillance and diagnosis scenarios. A fully covered 2D panorama surface view of an oil and gas field may need a very large number of airborne images or video frames. Particularly, to reconstruct a dense 3D surface map for some specific locations of the oil and gas plantation (1 acre for instance), thousands of high resolution images may be needed. The generated 3D map can be applied in augmented reality of the surface facility. Metadata and subsurface information can be presented through an intuitive way of asset understanding.

The lack of computationally efficient analysis tools has become a bottleneck for transforming the 2D imagery data into panorama views and 3D space.

### SUMMARY

In accordance with some embodiments of the disclosed subject matter, a method and a system for controlling unmanned aerial vehicles (UAVs) using smart navigation are provided herein. One aspect of the disclosed subject matter provides a smart system for creating a 2D panorama surface view of an oil and gas field, as well as generating a 3D visible and thermal map based on aerial images via cloud and high-performance graphical processing units (GPUs) and/or high-performance clusters (HPCs).

In accordance with some embodiments of the disclosed subject matter, a system for displaying a 3D visible and thermal map in augmented reality is provided. In some embodiments, a method and a smart system for detecting multiple objects from aerial images is provided. The method includes allocating image memory for parallel computation of a plurality of real-time input images by a group of GPUs or HPCs, performing, by registration kernels of the plurality of GPUs/HPCs, a fast pair-wise registration process to register the plurality of images, and performing, by mask setting kernels of the plurality of GPUs/HPCs, a mask setting process for the registered images to stitch the registered images into combined output images.

The method also includes performing, by background generation kernels of the plurality of GPUs/HPCs, a background generation process that incorporates the combined output images to generate background images using a median filter, performing, by foreground generation kernels of the plurality of GPUs/HPCs, a foreground generation process that incorporates the combined output images to generate foreground images, and performing, by classification kernels of the plurality of GPUs/HPCs, a deep learning classification process that classifies a plurality of objects identified in the real-time input images. Still further, the method includes generating a visualization including a 3D construction and 2D panorama image of an oil and gas environment surface that includes the combined output images, background images, foreground images and classified objects, and identifying and classifying one or more targets of interest using the generated visualization.

In some embodiments, aerial input images are generated from a visible and infrared imagery system mounted on a smart UAV navigation system. The consecutive image frames are applied to generate the 2D surface panorama of the oil and gas field, as well as the augmented reality and thermal map reconstruction of specific areas of interest (oil pumps, oil tanks and pipelines).

In some embodiments, the fast pair-wise registration process is a Compute Unified Device Architecture (CUDA) based parallel computing infrastructure. The process includes performing a speeded up robust features extraction process for each image pair, performing a point matching process for each image pair, using a random sample consensus algorithm to remove outlier points from the plurality of image pairs, and performing a transformation estimation process of the images to generate pair-wise homography matrices.

In some embodiments, stitching the registered images is based on the pair-wise homography matrices generated from the transformation estimation process, where a number of threads per block is consistent with available shared memory of the GPUs/HPCs. In some embodiments, the point matching process is based on Brute-force or Flann methods. In some embodiments, the background generation process comprises a background setting step, an image averaging step, and a background extraction step, and is a parallelized process implemented based on the GPUs/HPCs using a specified data structure.

In some embodiments, the foreground generation process comprises a pixel value comparison step, a value assigning step, and a foreground extraction step. In some embodiments, the deep learning classification process comprises: training of a Convolution Neural Network (CNN) based on GPUs/HPCs device, classifying the anomaly situation (e.g. oil leak, flare, vent, suspicious pedestrians and vehicles) based on the foreground extraction, and monitoring the multiple objects on the visualization and classification images. In some embodiments, the methods herein further include generating an augmented reality interface through open source computer graphics library associated with the GPUs/HPCs and 3D visible and thermal map for understanding the asset.

Another aspect of the disclosed subject matter provides a system for detecting acoustic anomalies from a background audio acquisition. This includes implementing a microphone system to collect the background and environment noise and filter low-frequency noise. The system further classifies the low-frequency noise (distinguishing the normal from the anomaly) and triggers an alarm if the acoustic anomaly was detected.

Another aspect of the disclosed subject matter provides a system for detecting gas concentration at the site and thereby determine where people and assets are located and further determine their real-time status to minimize risk. To make this detection, a gas sensor is mounted on a UAV configured to perform the gas concentration detection. The system may be designed to ring an alarm if the gas sensor reveals vulnerabilities. Other aspects of the disclosed subject matter can be understood by those skilled in the art in light of the description, the claims, and the drawings of the present disclosure.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements. It should be noted that the following drawings are merely examples for illustrative purposes according to various disclosed embodiments and are not intended to limit the scope of the present disclosure.

FIG. 1 illustrates an embodiment of a computing architecture configured to perform surveillance and diagnosis of an oil and gas surface environment via UAV;

FIG. 2 illustrates an exemplary flowchart of the smart visualization and surveillance system for oil and gas surface environment with various embodiments of disclosed subject matter;

FIG. 3 illustrates a flowchart of background generation and foreground generation processes in accordance with some embodiments of the disclosed subject matter;

FIG. 4 illustrates an exemplary process of pair-wise registration, and mask-setting in accordance with various embodiments of disclosed subject matter;

FIG. 5 illustrates visualization of an exemplary pair-wise SURF point matching in accordance with some embodiments of the disclosed subject matter;

FIG. 6 illustrates an exemplary process of pair-wise registration kernel in GPUs/HPCs and homography matrices multiplication in accordance with some embodiments of the disclosed subject matter;

FIG. 7 illustrates an exemplary highly parallel computation infrastructure of foreground generation in accordance with various embodiments of the present disclosure;

FIG. 8 illustrates a schematic diagram of hardware of an exemplary Cloud system for processing the audio and video input from the smart UAV navigation system in accordance with some embodiments of the disclosed subject matter;

FIG. 9 illustrates visualization of an exemplary augmented reality scenario and 3D visible map reconstruction of a small oil field in accordance with some embodiments of the disclosed subject matter;

FIG. 10 illustrates visualization of an exemplary background image in accordance with some other embodiments of the disclosed subject matter;

FIG. 11 illustrates visualization of an exemplary registered raw image in accordance with some other embodiments of the disclosed subject matter;

FIG. 12 illustrates visualization of an exemplary foreground image in accordance with various embodiments of present disclosure;

FIG. 13 illustrates visualization of an exemplary vehicle and human classification and tracking image in accordance with various embodiments of present disclosure;

FIG. 14 illustrates an embodiment of an apparatus including a UAV with a computer system configured to perform surveillance and diagnosis of an oil and gas surface environment;

FIG. 15 illustrates a visualization of an example 2D panorama image with high resolution of 4335×5887, composited from 50 images in a resolution of 2704×1520;

FIG. 16 illustrates a visualization of an example 2D panorama image with high resolution of 6290×5916, composited from 12 images in a resolution of 4000×3000; and

FIG. 17 illustrates visualization of an example 3D reconstruction of a group of real oil well facilities in the state of Louisiana.

### DETAILED DESCRIPTION

For those skilled in the art to better understand the technical solution of the disclosed subject matter, reference will now be made in detail to exemplary embodiments of the disclosed subject matter, which are illustrated in the accompanying drawings. Wherever possible, the same reference numbers will be used throughout the drawings to refer to the same or like parts.

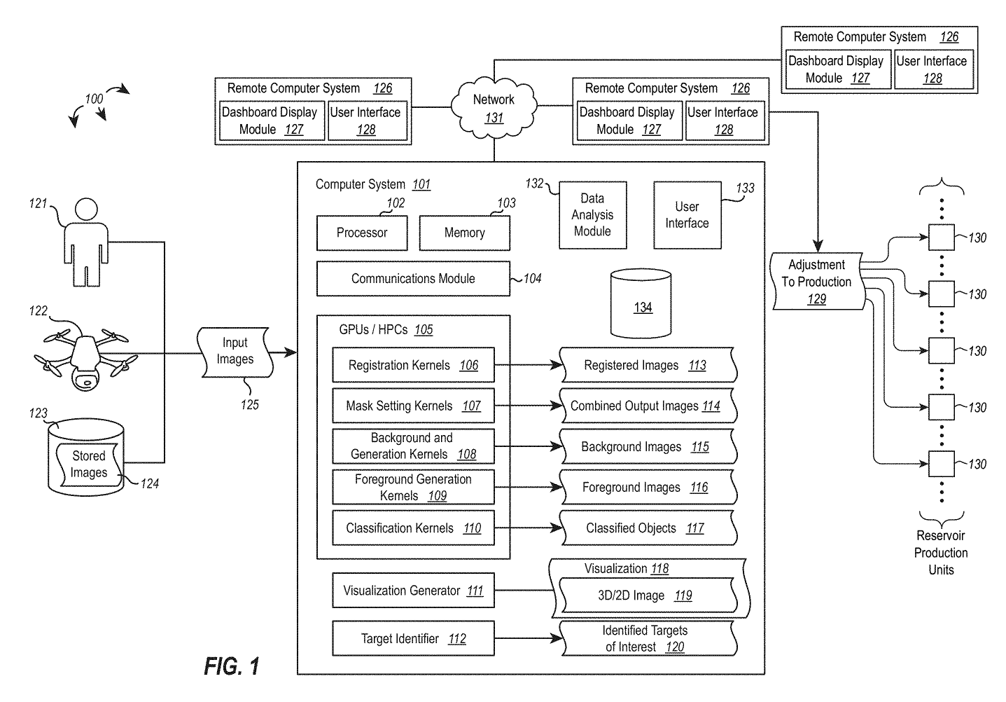

FIG. 1 illustrates a computing architecture 100 that is configured to perform surveillance and diagnosis of an oil and gas surface environment using a UAV. The computing architecture includes modules and components for performing different types of functionality. For instance, the computing architecture 100 includes a computer system 101 having at least one hardware processor 102 and system memory 103. The memory 103 may be physical system memory, which may be volatile, non-volatile, or some combination of the two. The term “memory” may also be used herein to refer to non-volatile mass storage such as physical storage media. If the computing system is distributed, the processing, memory and/or storage capability may be distributed as well.

As used herein, the term “executable module” or “executable component” can refer to software objects, routings, or methods that may be executed on the computing system. The different components, modules, engines, and services described herein may be implemented as objects or processes that execute on the computing system (e.g., as separate threads).

In the description that follows, embodiments are described with reference to acts that are performed by one or more computing systems. If such acts are implemented in software, one or more processors of the associated computing system that performs the act direct the operation of the computing system in response to having executed computer-executable instructions. For example, such computer-executable instructions may be embodied on one or more computer-readable media that form a computer program product. An example of such an operation involves the manipulation of data. The computer-executable instructions (and the manipulated data) may be stored in the memory 103 of the computer system 101. Computer system 101 may also contain communication channels, as described below, that allow the computer system 101 to communicate with other message processors over a wired or wireless network.

Embodiments described herein may comprise or utilize a special-purpose or general-purpose computer system that includes computer hardware, such as, for example, one or more processors and system memory, as discussed in greater detail below. The system memory may be included within the overall memory 103. The system memory may also be referred to as “main memory”, and includes memory locations that are addressable by the at least one processing unit 102 over a memory bus in which case the address location is asserted on the memory bus itself. System memory has been traditionally volatile, but the principles described herein also apply in circumstances in which the system memory is partially, or even fully, non-volatile.

Embodiments within the scope of the present invention also include physical and other computer-readable media for carrying or storing computer-executable instructions and/or data structures. Such computer-readable media can be any available media that can be accessed by a general-purpose or special-purpose computer system. Computer-readable media that store computer-executable instructions and/or data structures are computer storage media. Computer-readable media that carry computer-executable instructions and/or data structures are transmission media. Thus, by way of example, and not limitation, embodiments of the invention can comprise at least two distinctly different kinds of computer-readable media: computer storage media and transmission media.

Computer storage media are physical hardware storage media that store computer-executable instructions and/or data structures. Physical hardware storage media include computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the invention.

Transmission media can include a network and/or data links which can be used to carry program code in the form of computer-executable instructions or data structures, and which can be accessed by a general-purpose or special-purpose computer system. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIC”), and then eventually transferred to computer system RAM and/or to less volatile computer storage media at a computer system. Thus, it should be understood that computer storage media can be included in computer system components that also (or even primarily) utilize transmission media.

Computer-executable instructions comprise, for example, instructions and data which, when executed at one or more processors, cause a general-purpose computer system, special-purpose computer system, or special-purpose processing device to perform a certain function or group of functions. Computer-executable instructions may be, for example, binaries, intermediate format instructions such as assembly language, or even source code.

Those skilled in the art will appreciate that the principles described herein may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The invention may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

Those skilled in the art will also appreciate that the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

Still further, system architectures described herein can include a plurality of independent components that each contribute to the functionality of the system as a whole. This modularity allows for increased flexibility when approaching issues of platform scalability and, to this end, provides a variety of advantages. System complexity and growth can be managed more easily through the use of smaller-scale parts with limited functional scope. Platform fault tolerance is enhanced through the use of these loosely coupled modules. Individual components can be grown incrementally as business needs dictate. Modular development also translates to decreased time to market for new functionality. New functionality can be added or subtracted without impacting the core system.

The computer system 101 may further include a communications module 104. The communications module 104 may include any number of receivers, transmitters, transceivers, modems, radios or other communication devices. The radios may include, for example, WiFi, Bluetooth, cellular, GPS or other types of radios. These radios may be configured to receive data from (or transfer data to) other computer systems or other users. For instance, the communications module may be configured to receive input images 125 from a UAV 122 (or alternatively referred to as a drone herein). Additionally, or alternatively, the input images 125 may be received from a user 121 (i.e. from a user's mobile device), or from a data store 123 having stored images 124.

Computing architecture 100 may also include one or more remote computers 126 that permit a user, team of users, or multiple parties to access information generated by main computer system 101. For example, each remote computer 126 may include a dashboard display module 127 that renders and displays dashboards, metrics, or other information relating to reservoir production, alarms, anomaly detection, etc. Each remote computer 126 may also include a user interface 128 that permits a user to make adjustment to production 129 by reservoir production units 130. Each remote computer 126 may also include a data storage device (not shown).

Individual computer systems within computer architecture 100 (e.g., main computer system 101 and remote computers 126) can be connected to a network 131 using the communications module 104, such as, for example, a local area network (“LAN”), a wide area network (“WAN”), or even the Internet. The various components can receive and send data to each other, as well as other components connected to the network 131. Networked computer systems (i.e. cloud computing systems) and computers themselves constitute a “computer system” for purposes of this disclosure.

Networks facilitating communication between computer systems and other electronic devices can utilize any of a wide range of (potentially interoperating) protocols including, but not limited to, the IEEE 802 suite of wireless protocols, Radio Frequency Identification (“RFD”) protocols, ultrasound protocols, infrared protocols, cellular protocols, one-way and two-way wireless paging protocols, Global Positioning System (“GPS”) protocols, wired and wireless broadband protocols, ultra-wideband “mesh” protocols, etc. Accordingly, computer systems and other devices can create message related data and exchange message related data (e.g., Internet Protocol (“IP”) datagrams and other higher layer protocols that utilize IP datagrams, such as, Transmission Control Protocol (“TCP”), Remote Desktop Protocol (“RDP”), Hypertext Transfer Protocol (“HTTP”), Simple Mail Transfer Protocol (“SMTP”), Simple Object Access Protocol (“SOAP”), etc.) over the network.

Computer systems and electronic devices may be configured to utilize protocols that are appropriate based on corresponding computer system and electronic device on functionality. Components within the architecture can be configured to convert between various protocols to facilitate compatible communication. Computer systems and electronic devices may be configured with multiple protocols and use different protocols to implement different functionality. For example, a UAV 122 at an oil well might transmit data via infrared or other wireless protocol to a receiver (not shown) interfaced with a computer, which can then forward the data via fast Ethernet to main computer system 101 for processing. Similarly, the reservoir production units 130 can be connected to main computer system 101 and/or remote computers 126 by wire connection or wireless protocol.

Input images 125 may be processed by one or more GPUs or HPCs 105. The GPUs/HPCs 105 may be part of the computer system 101, or may be physically located in another location. For example, the GPUs/HPCs may be distributed over a wide geographic region, but may be configured to work together on a common task. Substantially any number of GPUs/HPCs may be used in the embodiments herein. The GPUs/HPCs 105 may have different kernels that are optimized to perform different tasks. For example, the registration kernel may be configured to perform a registration task that generates registered images 113. The mask setting kernels 107 may perform a mask setting task that combines the output images 114 into a single image or into a series of stitched images that each include a plurality of images. These images may be taken by a UAV at an oil field, for example, or other location such as an oil processing facility.

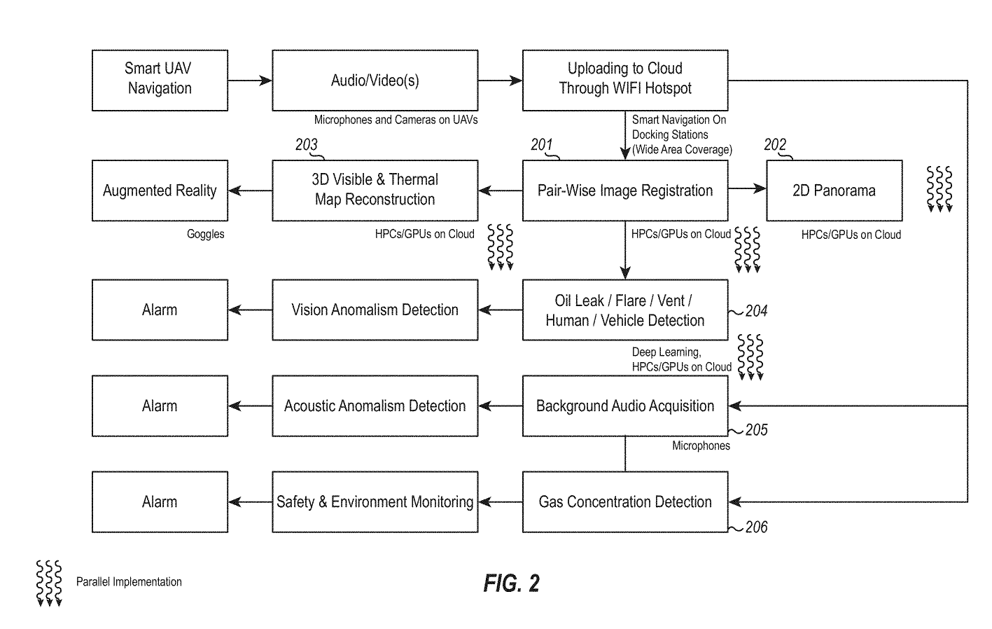

Still further, the GPUs/HPCs 105 may include background generation kernels 107 that are configured to generate background images 115 using the registered images 113. Foreground generation kernels 109 are configured to generate foreground images 116, and classification kernels 110 are configured to generate classified objects 117 identified in the images. The computer system 101 also includes a visualization generator 111 that generates visualizations 118 of a given site or location. The visualizations may include 3D representations and/or 2D panorama images that provide multiple details about an oil field or other site. A target identifier 112 analyzes the visualization to identify targets of interest 120. These targets of interest may be people, oil seeps, gas leaks, or other items. Each of these aspects will be described further below with regard to FIGS. 2-17.

In one embodiment, a method is provided for smart surveillance and diagnosis of an oil and gas surface environment via unmanned aerial vehicle. The method includes allocating image memory for parallel computation of a plurality of real-time input images 125 by a group of graphics processing units (GPUs) or high-performance clusters (HPCs) 105. The method next includes performing, by registration kernels 106 of the plurality of GPUs/HPCs 105, a fast pair-wise registration process to register the plurality of images, performing, by mask setting kernels 107 of the plurality of GPUs/HPCs, a mask setting process for the registered images 113 to stitch the registered images into combined output images 114, performing, by background generation kernels 108 of the plurality of GPUs/HPCs, a background generation process that incorporates the combined output images 114 to generate background images 115 using a median filter.

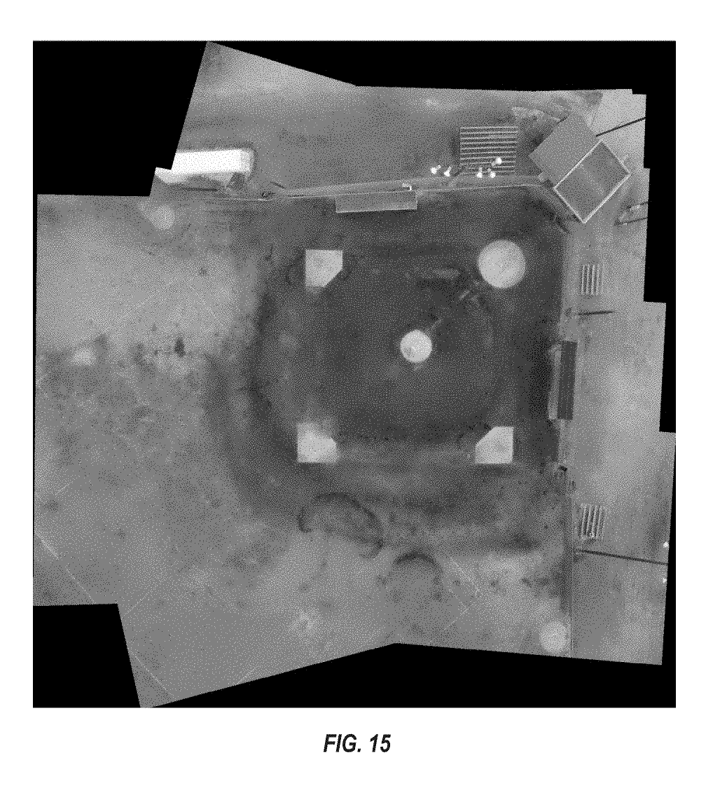

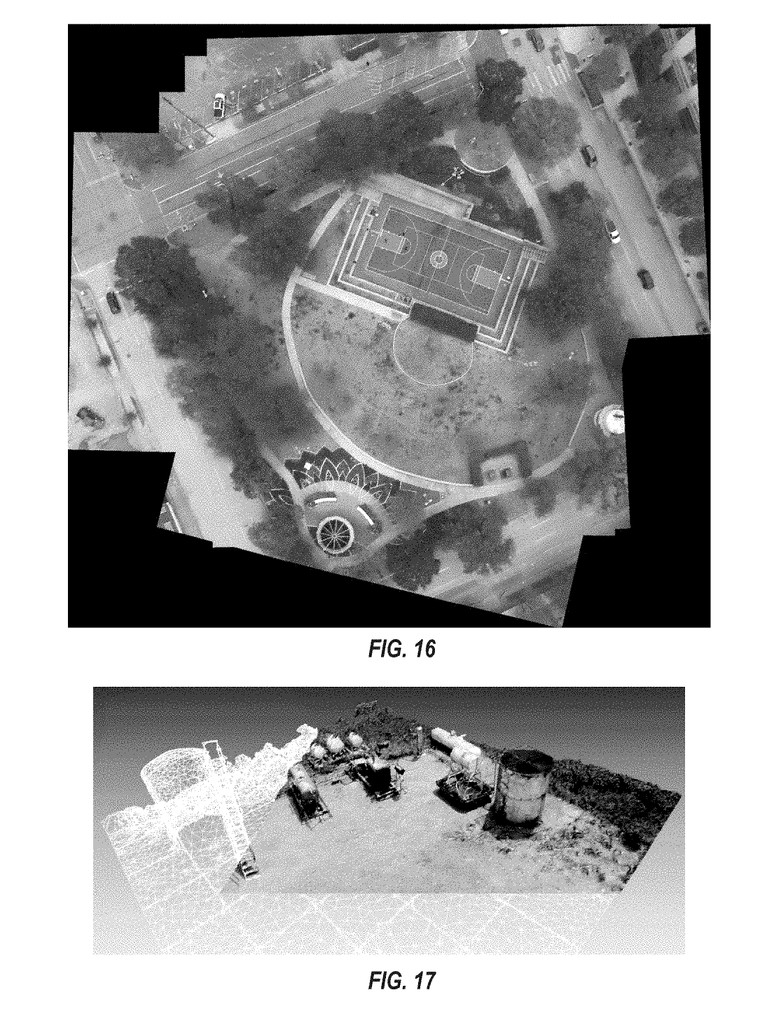

Still further, the method includes performing, by foreground generation kernels 109 of the plurality of GPUs/HPCs, a foreground generation process that incorporates the combined output images 114 to generate foreground images 116, performing, by classification kernels 110 of the plurality of GPUs/HPCs, a deep learning classification process that classifies a plurality of objects 117 identified in the real-time input images 125, generating a visualization 118 including a 3D construction (such as FIG. 17) and 2D panorama image 119 (also, see FIGS. 15 and 16) 119 of the oil and gas environment surface that includes the combined output images, background images, foreground images and classified objects, and identifying and classifying one or more targets of interest 120 using the generated visualization 119.

Indeed, in accordance with various embodiments, the disclosed subject matter herein provides a method for surveying and diagnosing the surface of oil and gas plantation based on airborne imagery and acoustic datasets via UAV smart navigations and parallel computation in GPUs and/or HPCs. In accordance with some other embodiments, the disclosed subject matter provides a High-performance Computing based system to implement the disclosed method (e.g. computer system 101). In some embodiments, visible light cameras and/or thermal cameras are mounted and aligned on the UAVs. As such, the visible and thermal images captured by these two sources have may have minute rotation and translation differences. Smart navigated UAVs can capture visible light and thermal videos of an area the size of an oil and gas field at the same time. This system may use two or more cameras mounted on some form of a gimbal on an aircraft or blimp to capture a very large field on the ground, from about ten per second up to thirty per second. Persistent surveillance captures the same general area on the ground over a specified length of time.

In some embodiments, median background modeling is implemented via GPUs to address the high computation complexity of detecting multiple objects in the input images 125. To avoid a large memory requirement and provide high throughput of video frames, a fast pair-wise image registration and multiple targets detection infrastructure is provided using the GPUs/HPCs 105.

In some embodiments, an asynchronous multiple objects detection can be achieved by the disclosed high-performance computing system 101. For example, detection or classification of multiple objects of interest from image groups, frame 0 to frame 9 for instance, may be monitored based on asynchronous exchange of information between GPUs and CPUs and adaptive parallel computing implementation on the CPU-GPU system.

For example, detection or classification of multiple objects of interest may be performed within the framework of a Compute Unified Device Architecture (CUDA) parallel computing infrastructure for the application of monitoring and surveying. The disclosed method and system may innovate an operator-friendly GUI for observing and monitoring the detection results (e.g., in a form of boxes to highlight). The disclosed parallel-computing-based approach has a general purpose in the sense that the same idea can be applied and extended to other types of surveillance, such as flare and vent detection based on thermal images.

The computer system 101 may therefore include a data analysis module 132 programmed to generate metrics from the detection and/or classification of objects of interest. A user interface 133 provides interactivity with a user, including the ability to input data. Data storage device 134 can be used for long term storage of data and metrics generated from the data. According to one embodiment, the computer system 101 can provide for at least one of manual or automatic adjustment to production 129 by reservoir production units 130 (e.g., producing oil wells, water injection wells, gas injection wells, heat injectors, and the like, and sub-components thereof). Adjustments might include, for example changes in volume, pressure, temperature, well bore path (e.g., via closing or opening of well bore branches). The user interface 133 permits manual adjustments to production 129. The computer system 101 may, in addition, include alarm levels or triggers that, when certain conditions are met, provide for automatic adjustments to production 129.

When compared to applying the detection and visualization process in a central processing unit (CPU) alone, the application of parallel computing structures based on CUDA Basic Linear Algebra Subroutines (cuBLAS) can achieve a much faster outcome of detection and 3D visualization. Moreover, the obtained detection or classification results for the multiple objects may indicate that the parallel-based approach (e.g. deep learning) may provide dramatically improved, speeded-up performance in real-time and under realistic conditions.

Referring to FIG. 2, an example flowchart of a smart surveillance and diagnosis system for an oil and gas surface environment is provided herein. As illustrated, the method can be implemented by a system including multiple GPUs or HPCs on cloud servers. The data transfer may occur through WiFi hotspots on docking stations in the field, or via other wireless data transfers.

In some embodiments, the cloud server includes at least one GPU or HPC. In the example as shown in FIG. 2, GPUs/HPCs can be used to apply parallel image processing such as image registration (step 201), 2D Panorama view generation (step 202), 3D visible and thermal map reconstruction (step 303) and various anomaly situation detection (oil leak, flare, vent, human and vehicle detection in step 204). In some embodiments, multiple HPCs/GPUs can be used for rapidly manipulating memory to accelerate the image processing. Any suitable number of GPUs/HPCs can be used in the cloud system according to various embodiments of the present disclosure. As a result of detecting the anomaly situation, embodiments can perform manual and/or automatic adjustment of production as described above to remedy the detected situation. In some embodiments, alarms are generated to notify appropriate personnel to further investigate the anomaly situation.

In some embodiments, the input images (e.g. 125 from FIG. 1) are visible light and thermal images generated by UAV systems. For example, each input visible light image may have a pixel resolution higher than 12,000,000 pixels. Multiple targets of interest may be detected in each input image. In some embodiments, the input images are real-time images, analyzed by the computer system 101 as they are taken by the UAV 122. In one embodiment, the frame rate of the input images 125 can be equal or larger than 15 frames per second.

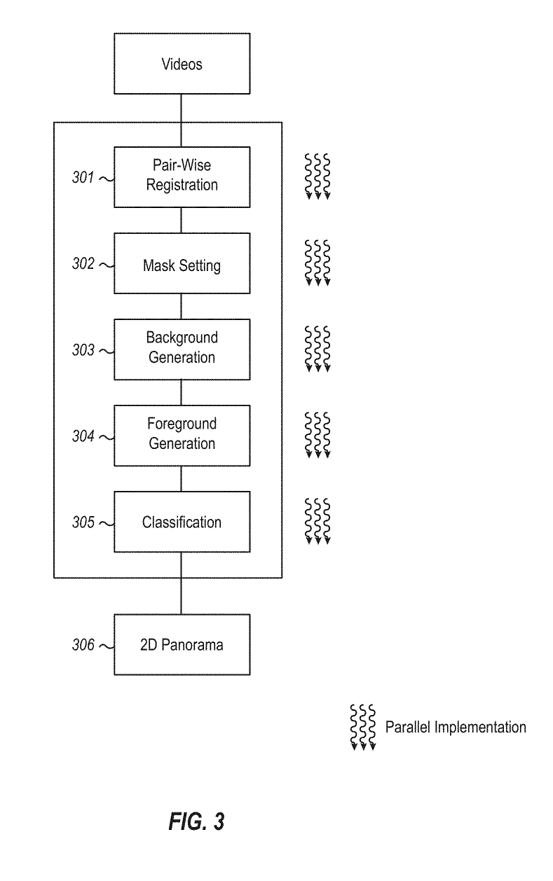

In some embodiments, the method further includes adaptive memory allocation corresponding to the size of pair-wise image groups associated with the GPUs. As a specific example of 2D panorama view generation, as illustrated in FIG. 3, the method steps can include pair-wise registration 301, mask setting 302, background generation 303, foreground generation 304 and classification 305. The 2D panorama generating step 306 will be further explained in greater detail below.

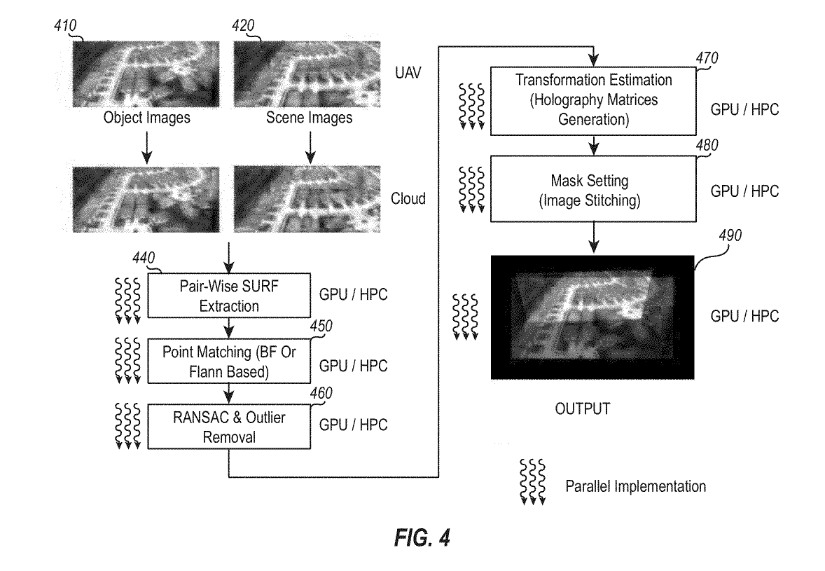

As a specific example of pair-wise registration 301, mask setting 302 and background generation 303, as illustrated in FIG. 4, two successive raw input images from UAV cameras include a front frame and a rear frame. The front frame can be an object image 410, and the rear frame can be a scene image 420. They are transferred to the cloud through WiFi hotspots or other appropriate communication systems.

Turning back to FIG. 2 and FIG. 3, at step 201 and 301, pair-wise image registration is performed by CUDA-based registration kernels of GPUs. In some embodiments, the pair-wise image registration kernel is configured to have one cluster or one GPU kernel processing two images at a specific time instant. In particular, when processing image data in a host CPU, certain memory space is allocated in the GPU. Then the data is copied from the host CPU to GPU, computation is performed in the GUP, then the data is transferred from the GPU to host CPU. Pair-wise image registration is a highly parallelized image processing. The multiple GPUs/HPCs (105) are very efficient to process the pair-wise images. The scene images are then warped to the coordinate of the object images based on the pair-wise transformation estimation. Each registration kernel may be configured to have at least one computing device process at least one pair of images at any specified time instant. Thus, for example, registration kernel 106 may be responsible for processing at least two images (i.e. one pair) at any given point in time.

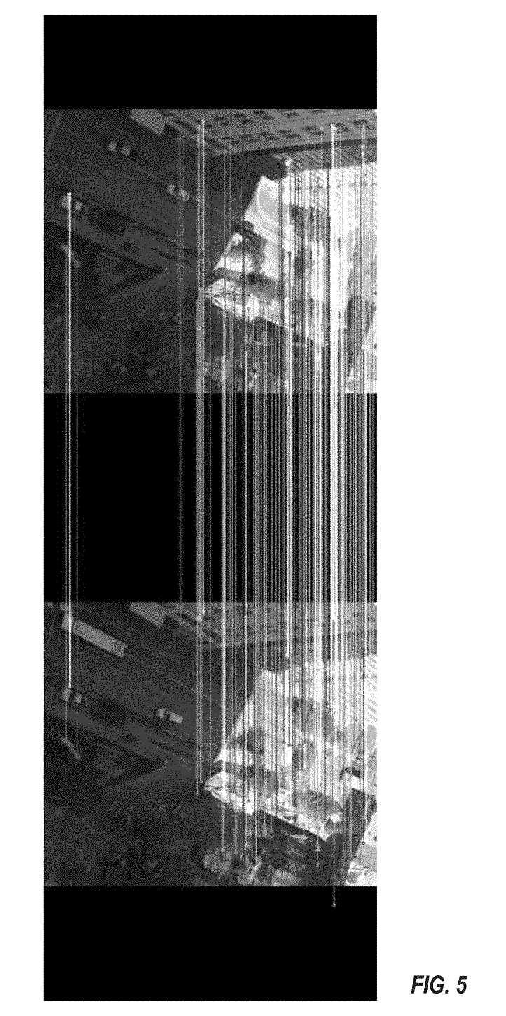

In some embodiments, the pair-wise image registration process performed in parallel by the multiple GPUs can include multiple steps described in the following. At 440 in FIG. 4, pair-wise speeded up robust features (SURF) extraction can be performed. In this step, point correspondences between two images of the same scene or object can be found. For example, some point of interest can be selected at distinctive locations in the image, such as corners, blobs, and T-junctions. Then, the neighborhood of every point of interest can be represented by a feature vector. Next, the feature vectors can be matched between the two images as can be seen in FIG. 5. In some embodiments, the matching is based on a distance between the vectors, e.g., the Mahalanobis or Euclidean distance.

In the example as shown in FIG. 4, the pair-wise SURF extraction 440 can be achieved by relying on integral images for image convolutions, and by building on the strengths of the leading existing detectors and descriptors. For example, a Hessian matrix-based measure can be used for the detector, and a distribution-based descriptor. At 450, point matching can be performed. In some embodiments, any suitable algorithm for performing fast approximate nearest neighbor searches in high-dimensional spaces can be used to realize the point matching. For example, the point matching can be Brute-force (BF) based, or FLANN based.

At 460, random sample consensus (RANSAC) and outlier removal can be performed. The RANSAC algorithm is an iterative method to estimate parameters of a mathematical model from a set of observed data which contains outliers by random sampling of observed data. Given a dataset whose data elements contain both inliers and outliers, RANSAC uses the voting scheme to find the optimal fitting result. Therefore, RANSAC can be performed as a learning technique to find outlier points from the results of the point matching. Then, the outlier points can be removed.

At 470, transformation estimation can be performed. In some embodiments, the transformation estimation can be applied among the object images and corresponding scene images to generate homography matrices. The estimated pair-wise homography matrices can be used to warp the scene images to the coordinate of the object images.

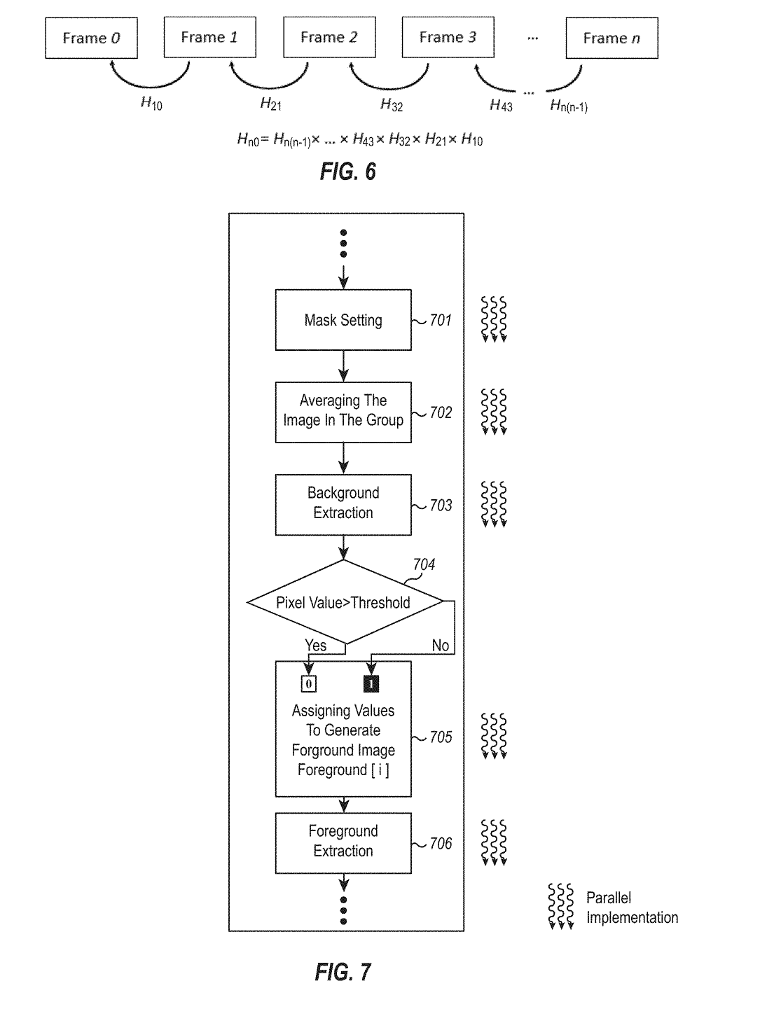

Referring to FIG. 6, an exemplary procedure of pair-wise transformation estimation and pair-wise image warping is shown in accordance with some embodiments. A scene image, which can be a frame behind the frame of the object image, can be paired with the object image. For instance, a pair-wise transformation estimation process can match the identified image features on frame 0 with frame 1 based on the homography matrix H₁₀. In the same way, pair-wise transformation estimation on frame n−1 with frame n is based on the homography matrix Hₙ₍ₙ₋₁₎. As a result, warping frame n to the world coordinate of frame 0 is based on the homography matrices multiplication Hₙ₀=Hₙ₍ₙ₋₁₎× . . . ×H₄₃×H₃₂×H₂₁×H₁₀. It should be noted that each pair-wise transformation estimation can be fed in an GPU/HPC kernel since they are time-independent operations between each other.

Accordingly, turning back to FIG. 3, the pair-wise image registration process 301 can include feature extraction 440, feature matching 450, random sample consensus (RANSAC) 460, and transformation estimation 470 in FIG. 4. Referring to step 480 in FIG. 4, pairs of the registered images can be collected on a mask via image stitching by kernels of the GPUs. In some embodiments, when launching the mask setting or image stitching kernel, the number of pairs of images is consistent with an available shared memory of the GPUs.

As can be seen in FIG. 4, the transformation estimation is applied among the object images and corresponding scene images. The estimated pair-wise homography matrices generated by the transformation estimation can be used to warp the scene images to the coordinate of the object images. Accordingly, a fused image 490 can be obtained by overlapping the object image 410 and the registered image 430. Returning back to FIG. 3, the fused image 430 can be used as an input of 303.

As illustrated in both FIGS. 3 and 4, the pair-wise registration and mask-setting processes are highly parallel. Considering the fact that GPUs are designed to operate concurrently, the pair-wise feature detection and description, the point matching, the RANSAC, the pair-wise transformation estimation, and the pair-wise image warping are all processed in GPUs/HPCs as can be seen in FIG. 6.

Turning back to FIG. 2 and FIG. 3, at step 202 and 306, 2D panorama, and background generation 303 are performed by background kernels of GPUs/HPCs 105. The background generation can be performed through a median filter (step 702) as can be seen in FIG. 7.

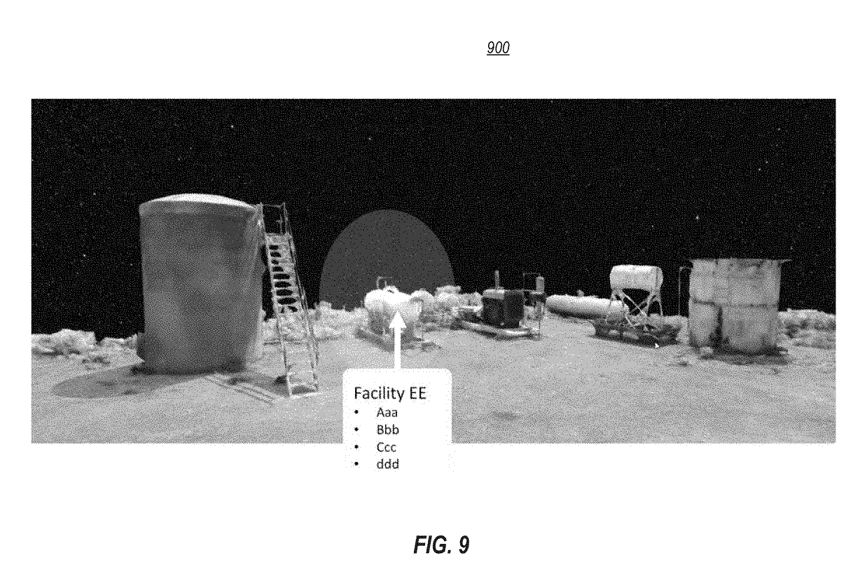

In some embodiments, each background generation kernel (steps 701-705) is configured to have one GPU/HPC integrated with a group of registered images at a time instant. For example, background generation can be performed for each group of multiple UAV images based on the stitched image by GPUs to generate one background image. As an illustrative example, referring to FIG. 9, a visualization of an exemplary background image 900 is shown accordance with some embodiments of the disclosed subject matter.

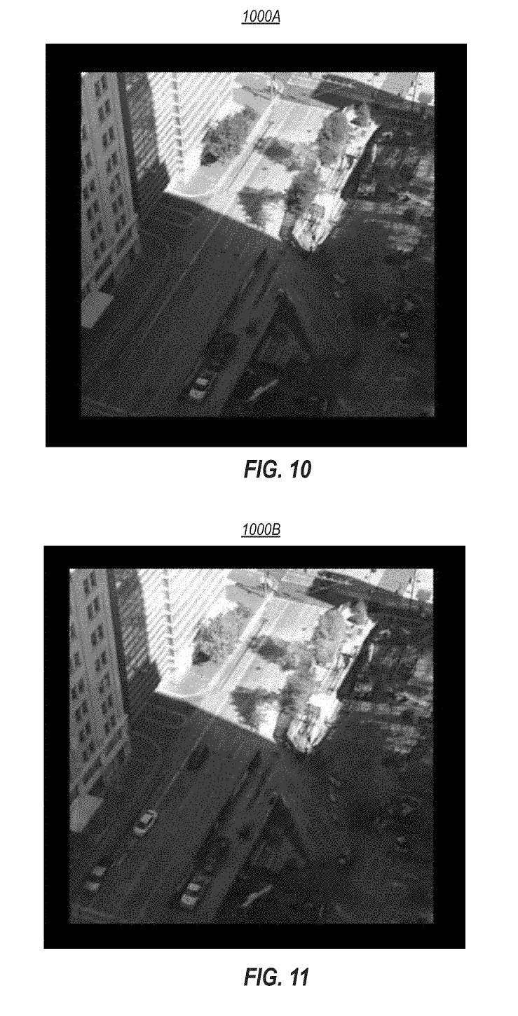

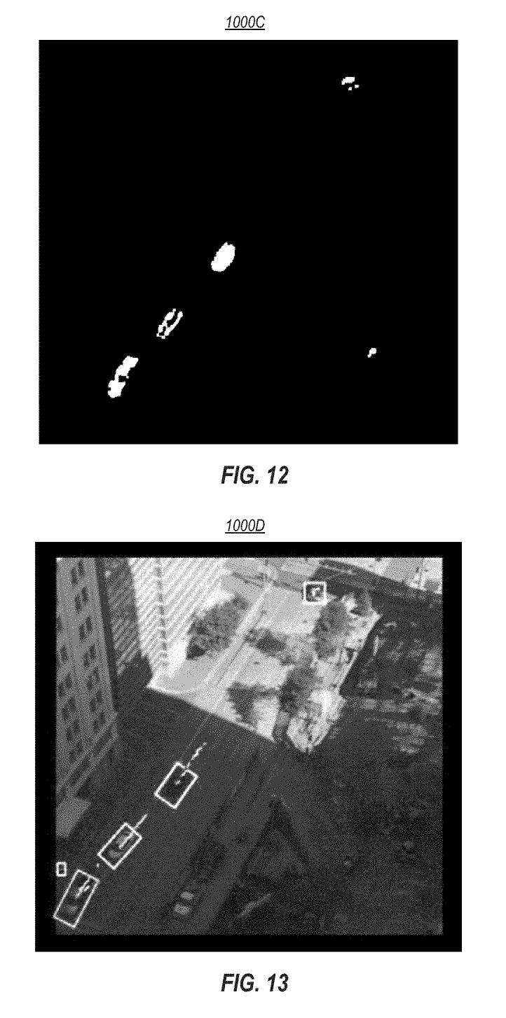

At step 706, foreground generation images are generated by foreground generation kernels of the GPUs/HPCs. The foreground generation can be performed based on image differences. In some embodiments, each foreground generation kernel is configured to have one GPU/HPC kernel process a group of registered images at a time instant. For example, foreground generation can be performed for each group of multiple UAV images based on the background image by GPUs to generate corresponding foreground image groups. As an illustrative example, referring to FIGS. 10-13, visualizations 1000A-1000D of an exemplary foreground image are shown in accordance with some embodiments of the disclosed subject matter. The highlighted objects (shown in irregular shapes (blobs) of FIG. 12) on the black background are the extracted foreground objects such as vehicles and/or pedestrians.

Referring to FIG. 7, a flowchart of background generation and the consecutive step foreground generation processes is shown in accordance with some embodiments of the disclosed subject matter. As illustrated, the background generation process can include mask setting at 701, averaging the image in the group at 702, and background extraction at 703. The background generation is a parallelized process implemented based on GPUs.

Noted that, CPU-based background generation in the smart visualization and surveillance system implements 2D traversal of the image sequences. This operational structure is computationally expensive, especially when the input sequences include large size images. For instance, the background extraction performed in the system may contains three nested FOR loops which are the size of height, the size of width and the size of the image groups.

As such, GPU computation can be applied to accelerate the background generation. The data structure dim3 in GPUs may be used to solve such problems such as memory allocation and parallel computation since the input are three-channel images in the smart visualization and surveillance system. This structure, used to specify the grid and block size, has three members [x, y and z] when compiling with certain programming languages such as C++. Thus, it is applied to store the image groups in device memory. Computation of a tile based in the data structure dim3 can be arranged, such that interactions in each row can be evaluated in a sequential order, while separate rows are evaluated in parallel in the GPUs.

As illustrated in FIG. 7, the foreground generation process can include pixel value comparison at 704, assigning values to generate foreground image at 705, and foreground extraction at 706. In some embodiments, the pixel values of output images 490 can be compared with a predetermined threshold value. For example, if a grey value of a pixel is larger than the predetermined threshold value (“yes” at step 704), the pixel can be determined as a part of the foreground image, and the pixel can be assigned as a value of “0” at step 705. On the other hand, if a gray value of a pixel is smaller than the predetermined threshold value (“no” at step 704), the pixel can be determined as a part of the background image, and the pixel can be assigned as a value of “1” at step 705.

The foreground generation is also a parallelized process implemented based on GPUs. CPU-based foreground generation has the same problem as the background generation. The only difference is that the outer loop is the size of image group, and the inner loops are size of height and the size of width. Rather than as background generation, the output of foreground generation is a group of binary (black and white) foreground images. Since the input are registered UAV images, for the construction convenience of the GPU implementation, the two inner loops are performed in GPUs. This computational architecture based on the IF-ELSE statement is quite efficient in a GPU/HPC platform.

Returning to FIG. 3, at step 305, classification can be performed by classification kernels of GPUs/HPCs. In some embodiments, the classification process can be performed based on deep learning networks (e.g. a Convolutional Neural Network). In some embodiments, probabilities or the confidence levels of each classified target of interest can be calculated based on CNN evaluation (Faster R-CNN or You Only Look Once (YOLO)). The classified objects of interest may include, for example, oil leak, flare, vent, vehicles and pedestrians, and can be updated in an online or on-the-fly manner.

For example, referring to FIG. 13, a visualization of an exemplary classification image is shown accordance with some embodiments of the disclosed subject matter. As illustrated, the classification image can be obtained based on the background image and foreground image shown in FIGS. 10 and 12 respectively. The final classification results of possible vehicle detection can be identified on the classification image. If anomalism is detected, the system will give alarms.

In some embodiments, a graphical user interface (GUI) can be generated for observing and monitoring the multiple objects detection in real-time during the image processing from the airborne video stream. For example, a real-time GUI can be generated for illustrating background images, foreground images, and classification images, such as the background image, foreground image, and classification image shown in FIGS. 10, 12 and 13.

Referring again to FIG. 2, step 205 provides a microphone system for detecting the acoustic anomalism from the background audio acquisition. It collects the background and environment noise (usually high-frequency). Low-frequency noise thus can be filtered. The low-frequency noise can be distinguished as normal or anomalism. If anomalism is detected, the system will give alerts. Step 206 provides a gas sensing system for detecting the gas concentration. This detection could detect where people and assets are located and their real-time status to minimize risk. The system will ring the alarm if the gas sensor reveals vulnerabilities.

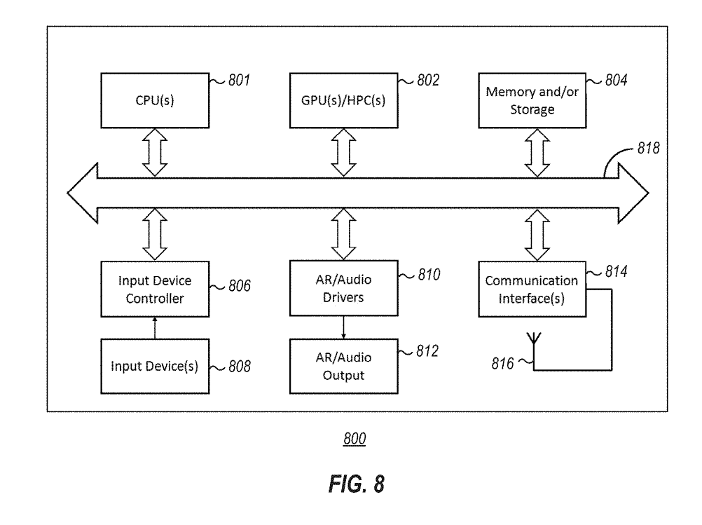

Referring to FIG. 8, a schematic diagram of hardware of an exemplary cloud system for multiple objects detection, augmented reality and audio detection is shown in accordance with some other embodiments of the disclosed subject matter.

As illustrated in the exemplary system hardware 800, such hardware can include at least one central processing unit (CPU) 801, multiple graphics processing units (GPUs) 802, memory and/or storage 804, an input device controller 806, an input device 808, AR/audio drivers 810, AR and audio output circuitry 812, communication interface(s) 814, an antenna 816, and a bus 818.

At least one central processing unit (CPU) 801 can include any suitable hardware processor, such as a microprocessor, a micro-controller, digital signal processor, array processor, vector processor, dedicated logic, and/or any other suitable circuitry for controlling the functioning of a general computer or special computer in some embodiments.

The multiple graphics processing units (GPUs) and high-performance clusters (HPCs) 802 include at least one graphics processing unit. The graphics processing unit can have any suitable form, such as dedicated graphics card, integrated graphics processor, hybrid form, stream processing form, general purpose GPU, external GPU, and/or any other suitable circuitry for rapidly manipulating memory to accelerate the processing of the audio signal, creation of 2D and 3D images in a frame buffer intended for output to a display and 3D reconstruction through structure from motion (SFM) technique in some embodiments.

In some embodiments, the at least one CPU 801 and the multiple GPUs/HPCs 802 can implement or execute various embodiments of the disclosed subject matter including one or more method, steps and logic diagrams. For example, as described above in connection with FIG. 6, the multiple GPUs/HPCs 802 can perform the multiple steps of pair-wise registration, mask setting, background generation, foreground generation, classification, etc. In some embodiments, the multiple GPUs 802 can implement the functions in parallel, as illustrated in FIG. 6. It should be noted that, the exemplary system hardware 800 is a GPU-CPU based system integrated with at least one CPU and multiple GPUs.

The steps of the disclosed method in various embodiments can be directly executed by a combination of the at least one CPU 801, and/or the multiple GPUs 802, and one or more software modules. The one or more software modules may reside in any suitable storage/memory medium, such as a random access memory, a flash memory, a read-only memory, a programmable read-only memory, an electrically erasable programmable memory, a register, etc. The storage medium can be located in the memory and/or storage 804. The at least one central processing unit (CPU) 801 and the multiple graphics processing units (GPUs) 802 can implement the steps of the disclosed method by combining the hardware and the information read from the memory and/or storage 804.

Memory and/or storage 804 can be any suitable memory and/or storage for storing programs, data, media content, comments, information of users and/or any other suitable content in some embodiments. For example, memory and/or storage 804 can include random access memory, read only memory, flash memory, hard disk storage, optical media, and/or any other suitable storage device.

Input device controller 806 can be any suitable circuitry for controlling and receiving input from one or more input devices 808 in some embodiments. For example, input device controller 806 can be circuitry for receiving input from a touch screen, from one or more buttons, from a voice recognition circuit, from a microphone, from a camera, from an optical sensor, from a gas sensor, from an accelerometer, from a temperature sensor, from a near field sensor, and/or any other suitable circuitry for receiving user input.

AR/audio drivers 810 can be any suitable circuitry for controlling and driving output to one or more augmented reality and audio output circuitries 812 in some embodiments. For example, AR/audio drivers 810 can be circuitry for driving an AR goggle, an LCD display, a speaker, an LED, and/or any other AR/audio device.

Communication interface(s) 814 can be any suitable circuitry for interfacing with one or more communication networks. For example, interface(s) 814 can include network interface card circuitry, wireless communication circuitry, and/or any other suitable circuitry for interfacing with one or more communication networks. In some embodiments, communication network can be any suitable combination of one or more wired and/or wireless networks such as the Internet, an intranet, a Wide Area network (“WAN”), a local-area network (“LAN”), a wireless network, a digital subscriber line (“DSL”) network, a frame relay network, an asynchronous transfer mode (“ATM”) network, a virtual private network (“VPN”), a WiFi network, a WiMax network, a satellite network, a mobile phone network, a mobile data network, a cable network, a telephone network, a fiber optic network, and/or any other suitable communication network, or any combination of any of such networks.

Antenna 816 can be any suitable one or more antennas for wirelessly communicating with a communication network in some embodiments. In some embodiments, antenna 816 can be omitted when not needed.

Bus 818 can be any suitable mechanism for communicating between two or more of components 802, 804, 806, 810, and 814 in some embodiments. Bus 818 may be an ISA bus, a PCI bus, an EISA bus, or any other suitable bus. The bus 818 can be divided into an address bus, a data bus, a control bus, etc. The bus 818 is represented as a two-way arrow in FIG. 8, but it does not mean that it is only one type bus or only one bus. Any other suitable components can be included in hardware 800 in accordance with some embodiments.

In some embodiments, the hardware of the exemplary system for smart surveillance based on multiple sources can be mounted onboard of an airplane. In some other embodiments, the hardware of the exemplary system for smart surveillance can be placed on cloud.

In addition, the flowcharts and block diagrams in the figures illustrate various embodiments of the disclosed method and system, as well as architectures, functions and operations that can be implemented by a computer program product. In this case, each block of the flowcharts or block diagrams may represent a module, a code segment, a portion of program code. Each module, each code segment, and each portion of program code can include one or more executable instructions for implementing predetermined logical functions. It should also be noted that, in some alternative implementations, the functions illustrated in the blocks be executed or performed in any order or sequence not limited to the order and sequence shown and described in the figures.

For example, two consecutive blocks may actually be executed substantially simultaneously where appropriate or in parallel to reduce latency and processing times, or even be executed in a reverse order depending on the functionality involved in. It should also be noted that, each block in the block diagrams and/or flowcharts, as well as the combinations of the blocks in the block diagrams and/or flowcharts, can be achieved by a dedicated hardware-based system for executing specific functions, or can be achieved by a dedicated system combined by hardware and computer instructions.

In some embodiments, any suitable computer readable media can be used for storing instructions for performing the processes described herein. For example, in some embodiments, computer readable media can be transitory or non-transitory. For example, non-transitory computer readable media can include media such as magnetic media (such as hard disks, floppy disks, and/or any other suitable media), optical media (such as compact discs, digital video discs, Blu-ray discs, and/or any other suitable optical media), semiconductor media (such as flash memory, electrically programmable read only memory (EPROM), electrically erasable programmable read only memory (EEPROM), and/or any other suitable semiconductor media), any suitable media that is not fleeting or devoid of any semblance of permanence during transmission, and/or any suitable tangible media. As another example, transitory computer readable media can include signals on networks, in wires, conductors, optical fibers, circuits, any suitable media that is fleeting and devoid of any semblance of permanence during transmission, and/or any suitable intangible media.

The provision of the examples described herein (as well as clauses phrased as “such as,” “e.g.,” “including,” and the like) should not be interpreted as limiting the claimed subject matter to the specific examples; rather, the examples are intended to illustrate only some of many possible aspects.

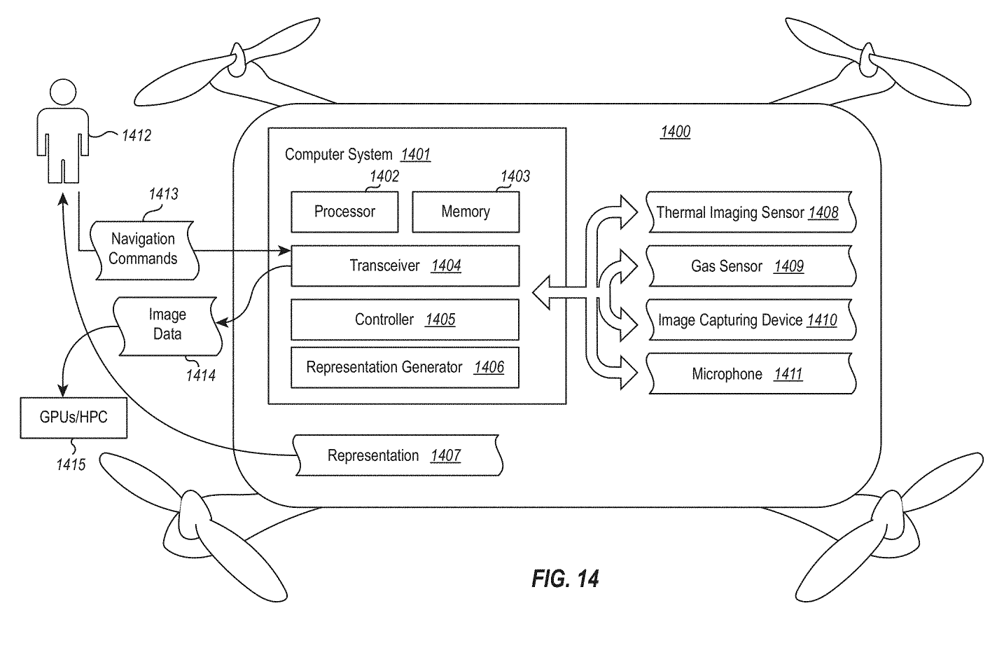

Turning now to FIG. 14, a system is provided for smart surveillance and diagnosis of an oil and gas surface environment. The system includes multiple elements including at least one unmanned aerial vehicle (UAV) 1400. The UAV 1400 may be any type or size of unmanned aerial vehicle flown by a local or remote pilot (e.g. 1412). The UAV 1400 receives navigation commands 1413 from the pilot and flies and performs other tasks according to these commands and/or any pre-programmed commands. The UAV 1400 includes at least one transceiver 1404 configured to communicate with a distributed computing system (e.g. GPUs/HPCs 1415). The transceiver 1404 is configured to transmit image data 1414 for a plurality of real-time input images to the distributed computing system. This allows the distributed computing system to process the image data 1414 using parallel computations. In some cases, at least a portion of the image processing may be performed on the processor 1402 and memory 1402 of the computer system 1401 on the UAV 1400. In other cases, the processor 1402 may merely be used to format the image data 1414 for transmission to the GPUs/HPCs 1415.

As noted above in FIG. 1, the distributed computing system (e.g. computer system 101) with GPUs/HPCs 105 may include registration kernels 106 for performing a fast pair-wise registration process to register the plurality of images 113. The GPUs/HPCs 105 may also include mask setting kernels 107 for performing a mask setting process for the registered images to stitch the registered images into combined output images 114, background generation kernels 108 for performing a background generation process using the combined output images to generate background images 115 using a median filter, foreground generation kernels 109 for performing a foreground generation process using the combined output images to generate foreground images 116 in a parallel manner, and classification kernels 110 for training a deep learning model to classify various objects of interest based on the foreground generation process. The distributed computing system 101 may also be configured for generating visualization classification images 119 based on a combination of the background images, foreground images and the identified targets of interest 120.

The real-time input images 125 may be generated from a smart UAV navigation system on the UAV. In some embodiments, the frame rate of the real-time input images 125 is at least 15 frames per second. The scale of each real-time input image may have a resolution having six orders of magnitude (i.e., above 1,000,000 pixels). In some cases, the objects that are to be identified in the input images 125 are oil leaks, flares, vents, vehicles, pedestrians, or other items that may be of interest on a hydrocarbon extraction site.

In at least some embodiments, the registration kernels 106 are configured to perform a fast pair-wise registration process using a Compute Unified Device Architecture (CUDA)-based parallel computing infrastructure. The CUDA pair-wise registration process includes performing a pair-wise speeded up features extraction process for each image pair, and performing a point matching process for each real-time input image using a random sample consensus algorithm to remove outlier points from the images. The CUDA pair-wise registration process also includes a transformation estimation process of the images to generate pair-wise homography matrices, as noted above. Each registration kernel is configured to have at least one computation device process a pair of images at a given time instant.

Continuing, the mask setting kernels 107 are configured to stitch the registered image pairs 113 based on the pair-wise homography matrices generated from the transformation estimation process. The number of threads per block is consistent with available shared memory of the plurality of GPUs. The background generation kernels 108 perform a background setting step, an image averaging step, and a background extraction step to generate background images 115. The background generation kernels 108 then implement a parallelized process using the plurality of GPUs based in a data structure such as a dim3 data structure.

The visualization generator 111 generates an augmented reality (AR) interface visualization 118 (such as for AR goggles or virtual reality (VR) goggles) using a computer graphics library associated with the GPUs/HPCs 1415. The visualization generator 111 may also generate a 3D visible and/or thermal map image 119 to aid in understanding the hydrocarbon extraction site surface environment, and/or may generate a graphical user interface using a computer vision library for a 2D panorama display image 119. The target identifier 112 may then identify and monitor multiple identified targets of interest 120 on the visualization classification images in real-time in the 3D AR/VR interface or on the 2D panorama display. These targets of interest may be any item that could affect the efficiency, production or safety of a hydrocarbon extraction site.

The UAV 1400 of FIG. 14 may further include various sensors for performing sensing tasks. For example, the UAV 1400 may include a microphone 1411 configured to detect audio waves. The microphone may be controlled by the processor 1402 of computer system 1401, or may be controlled via a separate controller 1405. The microphone may perform background audio acquisition while flying to identify sounds that may be out of the ordinary. If such an acoustic anomaly is detected, an alert process may be initiated by the computer system 1401 based on the acoustic anomaly detection. The microphone 1411 may be sensitive to noise, and may be capable of distinguishing acoustic anomalies from other background audio. The background and other environmental noise may be captured and stored and/or transmitted by the UAV for processing by the GPUs/HPCs 1415. The background noise is typically high-frequency audio data, and in many cases, the anomalies are manifest in low-frequency noises which can be filtered out and identified by the processor 1402 or the GPUs/HPCs 1415.

A gas sensor 1409 may also be included on the UAV 1400. The gas sensor may be used to detect gas concentrations or other gas-related anomalies. Upon detecting such anomalies, the computer system 1401 may initialize an alert process based on the gas concentration detection. As a result of this alert process, various users may be notified of the high gas concentration via a communication sent by the transceiver 1404. The alerts may be sent to users' mobile devices or other computer systems. The alerts may indicate that a gas-related anomaly has been identified, and may further recommend actions that should be taken by the user.

The UAV 1400 also includes imaging devices including a thermal imaging sensor 1408 and an image capturing device 1410. The thermal imaging sensor 1408 is configured to capture thermal images of a given location, showing which portions of the land are cooler or hotter. The image capturing device 1410 is configured to take visible-light images of the location. In some cases, infrared, ultraviolet or other imaging devices designed to capture or detect invisible light may also be used. The images may be captured and/or transmitted in real time back to the distributed computing system. The images may be taken at a frame rate of 15 frames per second (FPS), or at higher or lower FPS rates. The scale of each real-time image may have a resolution having at least six orders of magnitude. This allows users (or computer systems) to magnify images and drill down to find objects of interest. The images are also taken to scale, allowing the computer system 1401 (or a user) to calculate distance, volume or other measurements.

Deep learning, performed by the GPUs/HPCs 1415 may be used to classify the images, whether thermal or visible light images. The classification process may include training a deep learning model using labels via multi-fold convolution. Once the deep learning model has learned to identify objects of interest in an image (whether in the foreground or background), the deep learning model will be able to calculate probabilities or confidence levels for objects of interest found in the images. Thus, the deep learning models can not only identify images of interest, but can be trained to assign probabilities or confidence levels for the objects of interest that are found in the images.

In some embodiments, the GPUs/HPCs 1415 may be further configured to generate interfaces including 2D panorama interfaces and 3D virtual reality or augmented reality interfaces. These interfaces may be generated using a computer graphics library associated with the GPUs/HPCs. Augmented reality interfaces may include thermal data generated by a thermal imaging sensor, gas data generated by the gas sensor 1409 and other types of data. The generated interface may be used to monitor the identified multiple targets of interest in real-time. Thus, a user can use the interface to monitor oil leaks, flares, vents, vehicles, pedestrians, or other identified objects of interest.

In another embodiment, an apparatus is provided for surveying and maintaining an oil and gas surface environment. The apparatus includes an unmanned aerial vehicle (e.g. 1400 of FIG. 14) with a computer system 1401 mounted to it. The computer system 1401 includes at least one processor 1402, memory 1403, and a transceiver 1404. The computer system 1401 may also include some form of data storage (e.g. a flash drive or hard drive). In some cases, data stored in these UAV data stores may be automatically uploaded to the cloud and then deleted from local storage. The apparatus also includes a thermal imaging sensor 1408 mounted to the UAV that is communicatively connected to the computer system 1401. The thermal imaging sensor 1408 is configured to capture thermal readings over a specified area. Still further, the apparatus includes a microphone 1411 connected to the computer system 1401, which detects audio waves within range of the UAV.

The apparatus further includes a gas sensor 1409 mounted to the UAV that is communicatively connected to the computer system 1401. The gas sensor is configured to sense the presence of gases within range of the UAV. The apparatus further includes an image capturing device 1410 mounted to the UAV that is communicatively connected to the computer system 1401. The image capturing device 1410 is configured to capture images of land area within range of the UAV. The transceiver 1404 may be configured to receive navigation commands 1413 from a pilot 1412 or other user indicating where the UAV is to fly. In some cases, the UAV may be fully autonomous or semi-autonomous, allowing it to fly entirely or partially without human piloting input.

The transceiver 1404 may also receive sensor commands indicating when and how the thermal imaging sensor 1408, the gas sensor 1409, the image capturing device 1410, and the microphone 1411 (along with any other hardware) are to be operated during flight. Upon receiving the data from the various sensors and devices, the computer system 1401 may be configured to combine thermal imaging sensor data, gas sensor data, image data and/or audio data to create a combined representation 1407 of the oil and gas surface environment. The representation may change over time as new data is gathered by the UAV. The representation may also include comparisons between current data, previous day data, previous week or month data, previous year data, etc. Thus, the representation can show how thermal data, gas data, audio data or visible light data can change for a given area over time. Changes in temperature may indicate a flare, for example, and changes in gas concentration may indicate that venting is occurring at a given site.

Thus, the representation generator 1406 in the computer system 1401 may be configured to combine audio data detected by the microphone with thermal imaging sensor data, gas sensor data and image data to create a combined representation 1407 of the oil and gas surface environment. The combined representation may show where audio or gas anomalies were found, where objects of interest were identified in a foreground image, or where thermal anomalies exist on an oil and gas field. Objects of interest may be tagged in the images or in the combined representation 1407. Items such as wells, pumps, storage tanks, vehicles, humans, or other items may be tagged as normal or as problematic. Problematic items may be listed in alerts that are sent to interested parties.

Accordingly, methods and systems for smart oil and gas surface environment surveillance and diagnose via UAV and Cloud computation are provided. In the disclosed method and system, the 3D visualization and surveillance uses highly parallel algorithms to achieve a real-time performance.

Although the disclosed subject matter has been described and illustrated in the foregoing illustrative embodiments, it is understood that the present disclosure has been made only by way of example, and that numerous changes in the details of embodiment of the disclosed subject matter can be made without departing from the spirit and scope of the disclosed subject matter, which is only limited by the claims which follow. Features of the disclosed embodiments can be combined and rearranged in various ways. Without departing from the spirit and scope of the disclosed subject matter, modifications, equivalents, or improvements to the disclosed subject matter are understandable to those skilled in the art and are intended to be encompassed within the scope of the present disclosure.

We claim:

1. A method, implemented at a computer system comprising at least one processor, for smart surveillance and diagnosis of an oil and gas surface environment via unmanned aerial vehicle (UAV), comprising:
allocating image memory for parallel computation of a plurality of real-time input images by a group of graphics processing units (GPUs) or high-performance clusters (HPCs);
performing, by registration kernels of the plurality of GPUs/HPCs, a fast pair-wise registration process to register the plurality of images;
performing, by mask setting kernels of the plurality of GPUs/HPCs, a mask setting process for the registered images to stitch the registered images into combined output images;
performing, by background generation kernels of the plurality of GPUs/HPCs, a background generation process that incorporates the combined output images to generate background images using a median filter;
performing, by foreground generation kernels of the plurality of GPUs/HPCs, a foreground generation process that incorporates the combined output images to generate foreground images;
performing, by classification kernels of the plurality of GPUs/HPCs, a deep learning classification process that classifies a plurality of objects identified in the real-time input images;
generating a visualization including a 3D construction and 2D panorama image of the oil and gas environment surface that includes the combined output images, background images, foreground images and classified objects; and
identifying and classifying one or more targets of interest using the generated visualization.

2. The method of claim 1, wherein:
the plurality of real-time input images are generated from a smart UAV navigation system on an aircraft; and
the scale of each real-time input image has a resolution of at least six orders of magnitude.

3. The method of claim 1, wherein:
the fast pair-wise registration process is a Compute Unified Device Architecture (CUDA) based parallel computing infrastructure, and comprises:
performing a pair-wise speeded up robust features extraction process for each image;
performing a point matching process for each real-time input image;
using a random sample consensus algorithm to remove outlier points from the plurality of real-time input images; and
performing a transformation estimation process on each of the pair-wise images to generate pair-wise homography matrices.

4. The method of claim 3, wherein the mask setting process includes stitching the registered images using the pair-wise homography matrices generated from the transformation estimation process, wherein a number of threads per block is consistent with available shared memory of the plurality of GPUs or HPCs.

5. The method of claim 3, wherein the point matching process is based on Brute-force or Flann matching algorithms.

6. The method of claim 1, wherein each registration kernel is configured to have at least one computing device process at least one pair of images at a specified time instant.

7. The method of claim 1, wherein the background generation process:
comprises a background initialization step, an image averaging step, and a background extraction step; and
is a parallelized process implemented using the plurality of GPUs based in data structure dim3.

8. The method of claim 1, wherein the foreground generation process:
comprises a pixel value comparison step, a value assigning step, and a foreground extraction step.

9. The method of claim 1, wherein the deep learning classification process comprises:
training the deep learning model using labels via multi-fold convolution; and
calculating probabilities or confidence levels for the plurality of objects of interest based on the foreground generations.

10. The method of claim 1, further comprising:
generating an augmented reality interface using a computer graphics library associated with the GPUs/HPCs, wherein the augmented reality interface includes three dimensions and thermal data generated by a thermal imaging sensor;
generating a graphical user interface using a vision library for a 2D panorama display; and
monitoring the plurality of targets of interest on the visualization classification images in real-time on the 2D panorama display.

11. The method of claim 1, further comprising:
implementing a microphone system for detecting acoustic anomalies from background audio detected at the microphone;
collecting the background and environment noise; and
distinguishing the anomaly low-frequency noise from the background audio.

12. A system for smart surveillance and diagnosis of an oil and gas surface environment, the system comprising:
at least one unmanned aerial vehicle (UAV);
at least one transceiver configured to communicate with a distributed computing system, wherein the transceiver is configured to transmit image data for a plurality of real-time input images to the distributed computing system, allowing the distributed computing system to process the image data using parallel computations; and
the distributed computing system comprising a plurality of graphics processing units (GPUs) or high-performance clusters (HPCs), wherein the distributed computing system includes the following:
one or more registration kernels for performing a fast pair-wise registration process to register the plurality of images;
one or more mask setting kernels for performing a mask setting process for the registered images to stitch the registered images into combined output images;
one or more background generation kernels for performing a background generation process using the combined output images to generate background images using a median filter;
one or more foreground generation kernels for performing a foreground generation process using the combined output images to generate foreground images in a parallel manner; and
one or more classification kernels for training a deep learning model to classify a plurality of objects of interest based on the foreground generation process,
wherein the distributed computing system is further configured for generating visualization classification images based on a combination of the background images, foreground images and the plurality of targets of interest.

13. The system of claim 12, wherein:
the real-time input images are generated from a smart UAV navigation system on the UAV;
a scale of each real-time input image has a resolution of at least six orders of magnitude; and
the plurality of objects include at least one oil leak, flare, vent, vehicle or pedestrian.

14. The system of claim 12, wherein:
the registration kernels are configured to perform the fast pair-wise registration process using a Compute Unified Device Architecture (CUDA) based parallel computing infrastructure, by:
performing a pair-wise speeded up robust features extraction process for each image pair;
performing a point matching process for each real-time input image;
using a random sample consensus algorithm to remove outlier points from the plurality of images; and
performing a transformation estimation process of the images to generate pair-wise homography matrices;
wherein each registration kernel is configured to have at least one computation device process at least one pair of images at a given time instant.

15. The system of claim 14, wherein the mask setting kernels are configured for stitching the registered image pairs based on the pair-wise homography matrices generated from the transformation estimation process, wherein a number of threads per block is consistent with available shared memory of the plurality of GPUs.

16. The system of claim 11, wherein the background generation kernels are configured for:
performing a background setting step, an image averaging step, and a background extraction step; and
implementing a parallelized process using the plurality of GPUs based in a data structure dim3.

17. The system of claim 11, wherein the visualization kernel is further configured for:
generating an augmented reality interface using a computer graphics library associated with the GPUs/HPCs and a 3D visible and thermal map for understanding the oil and gas surface environment; and
generating a graphical user interface using a computer vision library for a 2D panorama display; and
monitoring the plurality of targets of interest on the visualization classification images in real-time on the 2D panorama display,
wherein the plurality of targets of interest include at least one oil leak, flare, vent, vehicle or pedestrian.

18. An apparatus for surveying and maintaining an oil and gas surface environment, the apparatus comprising:
an unmanned aerial vehicle (UAV);
a computer system mounted to the UAV, the computer system including at least one processor, memory, and a transceiver;
a thermal imaging sensor mounted to the UAV and communicatively connected to the computer system, wherein the thermal imaging sensor is configured to capture thermal readings over a specified area;
a gas sensor mounted to the UAV and communicatively connected to the computer system, wherein the gas sensor is configured to sense the presence of one or more gases within range of the UAV; and
an image capturing device mounted to the UAV and communicatively connected to the computer system, wherein the image capturing device is configured to capture one or more images of an area within range of the UAV,
wherein the transceiver is configured to receive navigation commands indicating where the UAV is to fly, and further receive sensor commands indicating when and how the thermal imaging sensor, the gas sensor, and the image capturing device are to be operated during flight, and
wherein the computer system is configured to combine thermal imaging sensor data, gas sensor data and image data to create a combined representation of the oil and gas surface environment.

19. The apparatus of claim 18, further comprising a microphone configured to detect audio waves within range of the UAV.

20. The apparatus of claim 19, wherein the computer system is further configured to combine audio data detected by the microphone with the thermal imaging sensor data, gas sensor data and image data to create a combined representation of the oil and gas surface environment.
